(19) United States
(12) Patent Application Publication
(10) Pub. No.: US 2012/0288640 A1
(43) Pub. Date: Nov. 15, 2012

(54) BOVINE SERUM ALBUMIN (BSA)-DIAZIRINE, METHOD OF FORMING BSA-DIAZIRINE, AND METHOD OF SELECTIVELY FIXING BIOMATERIAL USING BSA-DIAZIRINE OF PHOTO-REACTIVE TYPE

(75) Inventor: Wan Joong KIM, Goyang (KR)

(73) Assignee: Electronics and Telecommunications Research Institute, Daejeon (KR)

(21) Appl. No.: 13/470,164

(22) Filed: May 11, 2012

(30) Foreign Application Priority Data
May 13, 2011 (KR) 10-2011-0045249

(51) Int. Cl. C07K 14/765; B05D 3/06

(52) U.S. Cl. 427/553; 530/363

(57) ABSTRACT

The present invention provides bovine serum albumin (BSA)-diazirine, a method of forming BSA-diazirine, and a method of selectively fixing biomaterial using thereof. The bovine serum albumin-diazirine may function as a blocker which prevents the non-specific binding, as well as a linker, which links the solid support and the biomaterial by photoreaction (by UV irradiation). Therefore, by using the bovine serum albumin-diazirine, it is possible to selectively fix the biomaterial.

### CROSS-REFERENCE TO RELATED APPLICATIONS

This U.S. non-provisional patent application claims priority under 35 U.S.C. §119 of Korean Patent Application No. 10-2011-0045249, filed on May 13, 2011, the entire contents of which are hereby incorporated by reference.

### BACKGROUND OF THE INVENTION

The present invention disclosed herein relates to bovine serum albumin (BSA)-diazirine, a method of forming BSA-diazirine, and a method of selectively fixing a biomaterial using BSA-diazirine of photo-reactive type.

Immobilization of photo-induced proteins by UV light is considered as a novel technique in the field of microarray sensors. Microarrays of biomaterials such as DNA or protein are important tools in the area of molecular diagnostics and proteomics. In particular, as compared with DNA microarrays, protein arrays have a wide range of applications in the fields such as proteome analysis, diagnosis of disease and quantification analysis.

In general, proteins can be deposited on a solid surface activated with glutaric aldehyde or NHS (N-Hydroxysuccinimide) by covalent or non-covalent bonding between the proteins and the surface or using a photoactivatable crosslinker such as diazirine. Among these methods, fixing protein by using a photoactivatable crosslinker may be advantageous since antibodies can be rapidly covalent-bonded to a solid surface. However, typical methods are not suitable for selective fixing to small areas. The reason for this is that most proteins can easily attach to a surface of any solid by physical adsorption within a minute regardless of whether the proteins are hydrophilic or hydrophobic. In addition, it is difficult to fix several types of antibodies to a micro chip using a spotting method because the spotting method is not suitable for micrometer applications.

In order to fix antibodies to small solid surfaces, it is important to reduce non-specific bindings for preventing the antibodies from binding to unwanted regions. There were attempts to prevent non-specific bindings to solid surfaces by inserting various polyethylene glycol (PEG) groups in an intermediate linker. However, these could not block non-specific bindings completely.

Therefore, in order to selectively fix proteins to small solid surfaces, it is necessary to develop a material capable of completely preventing non-specific bindings.

### SUMMARY OF THE INVENTION

The present invention provides a material capable of completely preventing non-specific bindings, and a method of preparing thereof.

The present invention also provides a method of selectively fixing a biomaterial to a solid surface by using a material capable of completely preventing non-specific binding.

Embodiments of the present invention provide bovine serum albumin-diazirine (BD). The bovine serum albumin-diazirine is a compound in which bovine serum albumin having an amine group is covalently bonded to at least one NHS-diazirine (N-Hydroxysuccinimide diazirine) having a structure of Formula 1.

[Formula 1]

In some embodiments, the NHS-diazirine is covalently bonded to the amine group of the bovine serum albumin. For example, 1 to 100 NHS-diazirines may bind to one bovine serum albumin. Specifically, about 60 NHS-diazirines may bind to one bovine serum albumin. The bovine serum albumin-diazirine formed by binding of the NHS-diazirine and the bovine serum albumin may have chemical/physical characteristics similar to bovine serum albumin.

In other embodiments of the present invention, there are provided methods for preparing the bovine serum albumin-diazirine. The methods may include allowing bovine serum albumin containing an amine group to react with Sulfo-SDAD (sulfosuccinimidyl 2-([4,4′-azipentanamido]ethyl)-1,3′-dithioproprionate) having a structure of Formula 2.

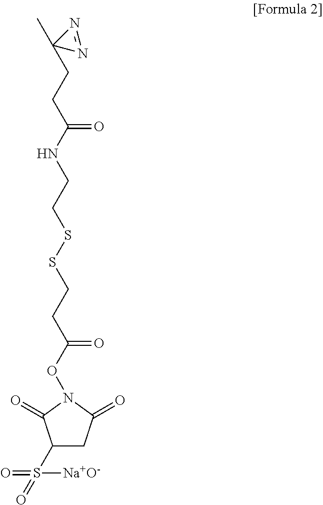

In some embodiments, the allowing of the bovine serum albumin to react with the Sulfo-SDAD may include stirring the bovine serum albumin and the Sulfo-SDAD. After the allowing of the bovine serum albumin to react with the Sulfo-SDAD, the methods may further include removing remaining bovine serum albumin and Sulfo-SDAD.

In still other embodiments of the present invention, there are provided methods for fixing a biomaterial, the method including: immobilizing the bovine serum albumin-diazirine on a substrate; and selectively fixing a biomaterial to the bovine serum albumin-diazirine by UV irradiation.

In some embodiments, the immobilizing of the bovine serum albumin-diazirine may include incubating the bovine serum albumin-diazirine on the substrate.

In other embodiments, the selective fixing of the biomaterial may include: selectively fixing protein A/G by UV irradiation; and fixing a biomaterial to the selectively fixed protein A/G.

In still other embodiments, in the selective fixing of the biomaterial, the UV irradiation may be performed for about 6 minutes to about 15 minutes.

In even other embodiments, in the selective fixing of the biomaterial, the UV irradiation may be performed with UV intensity of about 5 W/m² to about 18 W/m².

In yet other embodiments, the selective fixing of the biomaterial may include selectively casting UV light using a photomask.

In further embodiments, the methods may further include selectively casting UV light to form a micro pattern.

In still further embodiments, the biomaterial may be at least one selected from the group consisting of antibodies, membrane type serine proteases, and food toxins.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 is an illustration showing a process of manufacturing bovine serum albumin (BSA)-diazirine (BD) according to an embodiment of the present invention;

FIG. 2 is an illustration showing a process of fixing a biomaterial using BSA-diazirine (BD) according to an embodiment of the present invention;

FIG. 3 is a schematic diagram illustrating materials immobilized on a substrate in Example 2;

FIG. 4 is an FE-SEM image taken from a surface of the substrate in Example 2;

FIG. 5 is an FE-SEM image taken from a surface of a substrate in a control example;

FIG. 6 is a graph showing absorbance with respect to the concentration of protein A/G in Example 3;

FIG. 7 is a graph showing absorbance with respect to the intensity of UV irradiation in Example 4;

FIG. 8 is a graph showing absorbance with respect to the time of UV irradiation in Example 5;

FIG. 9 is a graph showing results of Example 6;

FIG. 10 is a schematic diagram illustrating a process of forming a micro pattern on a substrate by selective UV irradiation using a photomask according to Example 7; and

FIGS. 11A and 11B illustrate images taken from the micro pattern formed in Example 7.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art.

An embodiment of the present invention provides a bovine serum albumin-diazirine (BD). The bovine serum albumin-diazirine (BD) is a compound in which bovine serum albumin having an amine group is covalently bonded to at least one NHS-diazirine (N-Hydroxysuccinimide diazirine) having a structure of Formula 1.

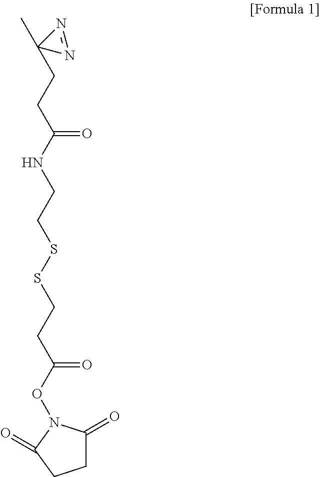

The NHS-diazirine is covalently bonded to the amine group of the bovine serum albumin. For example, 1 to 100 NHS-diazirines may bind to one bovine serum albumin. Specifically, about 60 NHS-diazirines may bind to one bovine serum albumin. The bovine serum albumin-diazirine (BD), which is formed by binding of NHS-diazirine and bovine serum albumin, may have chemical/physical characteristics similar to bovine serum albumin. In detail, the number of amino acid residues are about 585, and the molecular weight is about 66, 776, and the isoelectric point at 25° C. is about 4.7.

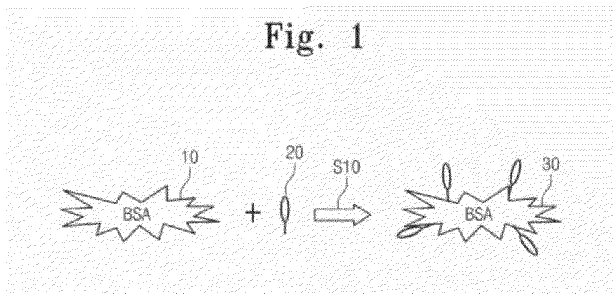

A method for preparing bovine serum albumin-diazirine (BD) will now be described with reference to FIG. 1. FIG. 1 is an illustration showing a process of preparing bovine serum albumin-diazirine (BD) according to an embodiment of the present invention.

Referring to FIG. 1, a bovine serum albumin-diazirine (BD) 30 is formed by reacting bovine serum albumin containing an amine group 10 with Sulfo-SDAD (Sulfosuccinimidyl 2-([4,4′-azipentanamido]ethyl)-1,3′-dithioproprionate) 20, represented by Formula 2.

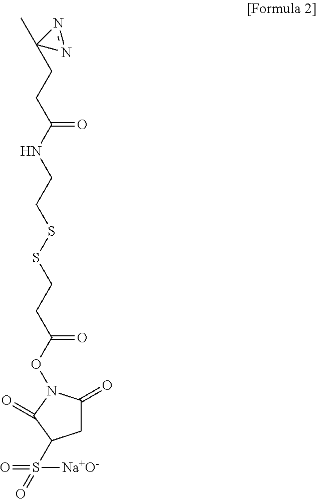

The reacting (S10) the bovine serum albumin 10 with the Sulfo-SDAD 20 may be performed by stirring the bovine serum albumin 10 and the Sulfo-SDAD 20. Remaining materials may be removed after the stirring, and the stirring and the removing may be repeated several times.

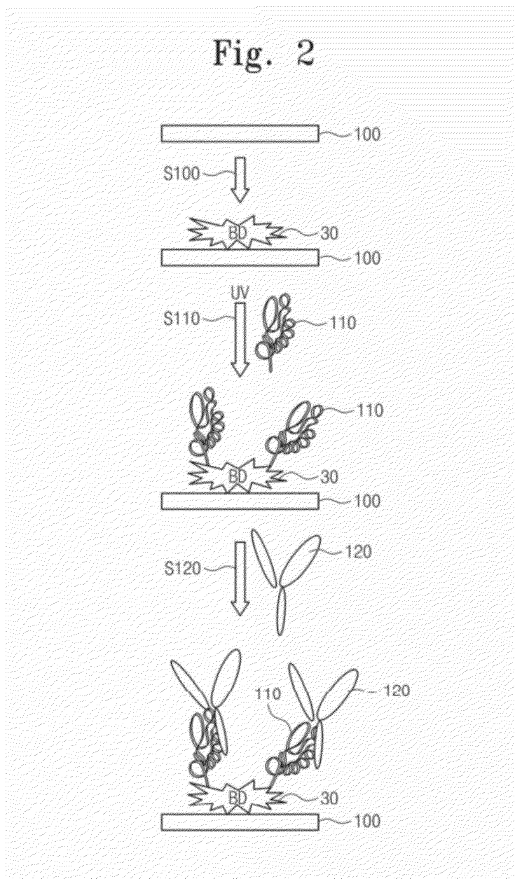

A method of fixing a biomaterial using the bovine serum albumin-diazirine (BD) 30 will now be described with reference to FIG. 2.

Referring to FIG. 2, the bovine serum albumin-diazirine 30 is fixed to a substrate 100 (in a first step S100). The first step S100 may be performed by incubating the bovine serum albumin-diazirine 30 on the substrate 100. Unfixed bovine serum albumin-diazirine 30 in the first step S100 may be removed by washing with 0.01% Tween 20, and dried by blowing N₂ across the substrate 100. The biomaterial may be fixed to the substrate 100 on which the bovine serum albumin-diazirine 30 is immobilized by placing the biomaterial on the substrate 100 and selectively casting UV light to the substrate 100. The biomaterial may be at least one selected from the group consisting of antibodies, membrane type serine proteases, and food toxins.

For example, protein A/G is fixed to the bovine serum albumin-diazirine 30 by adding the protein A/G and casting UV light (in a second step S110). The UV light may cast for about 1 minute to about 15 minutes. Owing to the UV light, nitrogen (N₂) is removed from the bovine serum albumin-diazirine 30, and sites of the bovine serum albumin-diazirine 30 from which nitrogen (N₂) is removed are activated so that protein A/G can be coupled to the sites. Antibodies 120 are fixed to the protein A/G by incubating the antibodies 120 on the substrate 100 where the protein A/G is coupled (in a third step S120). The fixation level of the antibodies 120 may be confirmed by using an enzyme-Linked immunosorbent assay (ELISA) and a silver enhancement method. Absorbance may be monitored to confirm the fixation level of the antibodies 120. In addition, metal particles may be bound to the antibodies 120. During the UV irradiation, only specific regions may be irradiated with UV light by using a photomask. In this way, antibodies 120 can be fixed to only specific regions of the substrate 100, and the metal particles can be bound to the antibodies 120. As a result, a micro pattern can be formed.

### Example 1

### Preparation of Bovine Serum Albumin (BSA)-Diazirine

BSA-diazirine was prepared by adding 1.7 mg of Sulfo-SDAD with 1 ml of 10% BSA in a micro-centrifuge tube (EP tube), followed by 2 hr of shaking. Non-reacted debris/impurities were removed by using Microcone (Ultracel YM-30) and 0.01% Tween-20. This process was repeated 3 times. The BSA-diazirine cleared of the debris/impurities was diluted in 1 ml of 1×PBS buffer (phosphate-buffered saline) and then stored at 4° C.

### Example 2

### Fixing Biomaterial to BSA-Diazirine by Selective UV Irradiation

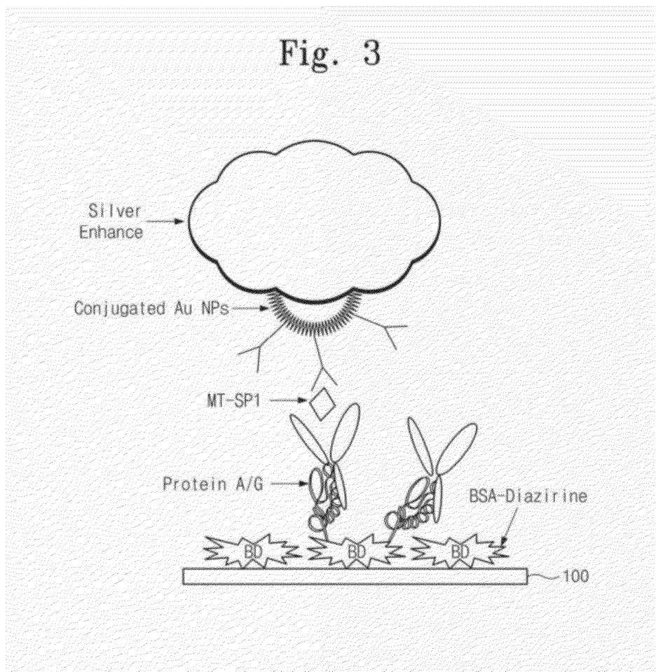

The BSA-diazirine prepared in Example 1 was immobilized on a 96-well plate substrate by incubating BSA-diazirine on the substrate for about 30 minutes. Unbound BSA-diazirine was removed using 0.01% Tween-20, and the substrate was dried by blowing N₂ gas across the substrate. Protein A/G was attached to BSA-diazirine by adding 50 μl of 1 mg/ml concentration of protein A/G to the substrate and casting UV light (wave length of 365 nm) for about 15 min at an intensity of 18 W/m². Unbound protein A/G was removed by washing the substrate with 0.01% Tween-20. Next, membrane type serine protease 1 (MT-SP1) was fixed to the substrate. In detail, 50 μl of 50 μg/ml concentration of monoclonal MT-SP1 antibody was added to the substrate and incubated for about 1 hour to attach the monoclonal MT-SP1 antibody to the protein A/G. ELISA was used to confirm fixation of the monoclonal MT-SP1 antibody. In detail, 50 μl of 100 ng/ml of MT-SP1 antigen was incubated on the substrate for about 1 hr, followed by an additional hour of incubation after adding poly MT-SP1 antibody conjugated with gold (Au) nanoparticles. Absorbance was measured by using ELISA reader after performing a silver enhancement method. The reproducibility of experimental data was confirmed by repeating Example 2 five times. The materials fixed to the substrate in Example 2 may be represented as shown in FIG. 3.

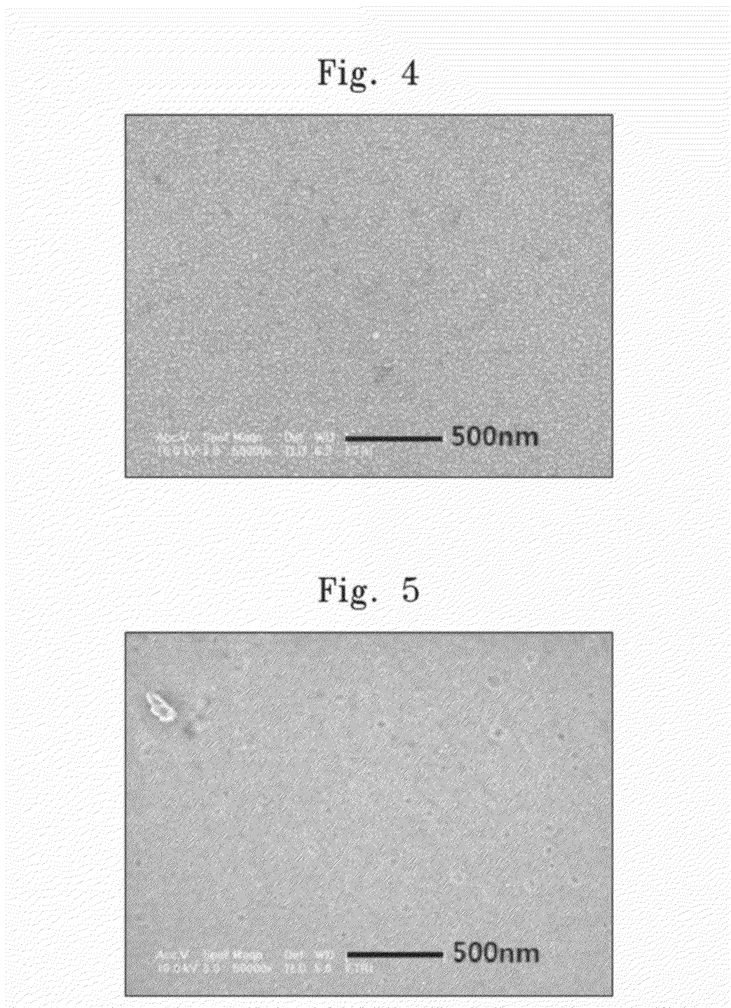

The absorbance measured in Example 2 was 2.25%. Also, an FE-SEM (field emission scanning electron microscope) image taken from the substrate is shown in FIG. 4. Referring to FIG. 4, the number of gold nanoparticles fixed to the substrate was approximately 830 μm².

### Control Example

### BSA-Diazirine Without UV Irradiation

BSA-diazirine was added on a 96-well plate substrate as in Example 2 and incubated for about 30 minutes to immobilize the BSA-diazirine on the substrate. Unbound BSA-diazirine was removed by washing with 0.01% Tween-20, and the substrate was dried by blowing N₂ gas across the substrate. 50 μl of 1 mg/ml concentration of protein A/G was added to the substrate. UV irradiation was not performed. Further experimental steps were performed like Example 2. In detail, the substrate was washed with 0.01% Tween-20. 50 μl of monoclonal MT-SP1 antibody at the concentration of 50 μg/ml was added to the substrate and incubated for 1 hour. ELISA was used to confirm the fixation of the monoclonal MT-SP1 antibody. In detail, 50 μl of MT-SP1 antigen at 100 ng/ml was incubated on the substrate for 1 hr, followed by an additional hour of incubation after adding poly MT-SP1 antibody conjugated with gold nanoparticles. The absorbance was measured by using an ELISA reader after performing a silver enhancement method. Absorbance was measured as 0.06%. Also, an FE-SEM image taken from the substrate is shown in FIG. 5. Referring to FIG. 5, the number of gold nanoparticles fixed to the substrate is approximately 12/μm². In summary, the low absorbance and small number of gold nanoparticles in the control group indicate that protein A/G did not attach to BSA-diazirine when there was no UV irradiation. The experiment was repeated five times to confirm the reproducibility of experimental data.

Comparison of the results of Example 2 and the control example indicates that protein A/G can be selectively fixed to BSA-diazirine depending on UV irradiation.

### Example 3

### Analysis of Fixation Level of Biomaterials Depending on the Concentration of Protein A/G

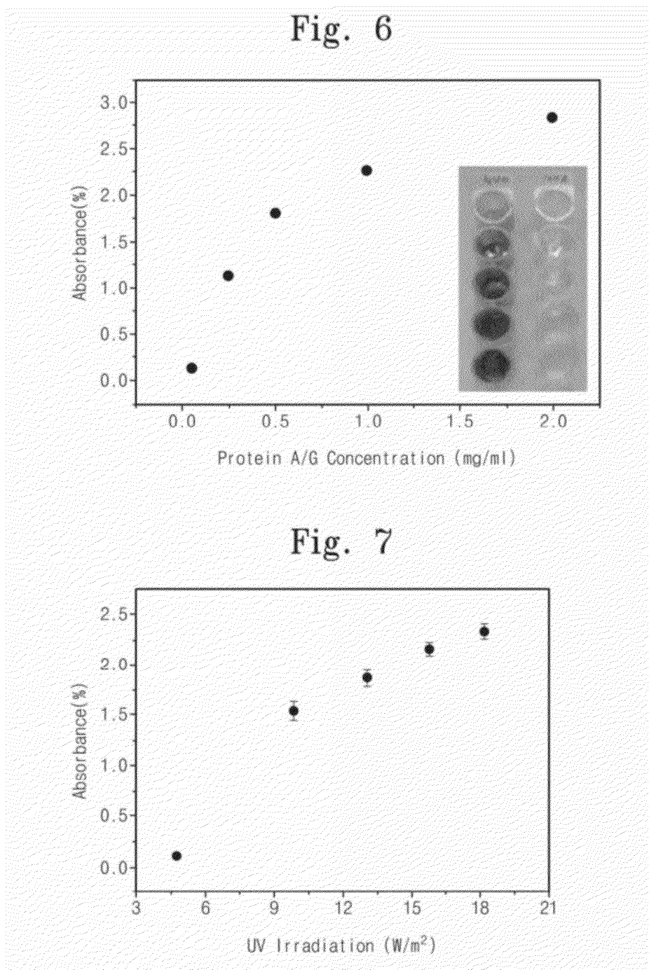

Five experimental samples were prepared to investigate the level of biomaterial fixation. In detail, BSA-diazirine was immobilized on five identical 96-well plate substrate (first to fifth substrates) by incubating the BSA-diazirine prepared in Example 1 on the substrates for 30 minutes. Unbound BSA-diazirine was removed by washing with 0.01% Tween-20, and the substrates were dried by blowing N₂ gas across the substrates. 50 μl of protein A/G at the concentration of 0.05 mg/ml was added to the first substrate. 50 μl of protein A/G at the concentration of 0.25 mg/ml was added to the second substrate. 50 μl of protein A/G at the concentration of 0.5 mg/ml was added to the third substrate. 50 μl of protein A/G at the concentration of 1 mg/ml was added to the fourth substrate. 50 μl of protein A/G at the concentration of 2 mg/ml was added to the fifth substrate. Protein A/G was attached to BSA-diazirine by casting UV light (wave length of 365 nm) for 15 minutes at an intensity of 18 W/m². Unbound protein A/G was removed by washing the substrate with 0.01% Tween-20. Next, membrane type serine protease 1 (MT-SP1) was fixed to the substrate. In detail, 50 μl of 50 μg/ml concentration of monoclonal MT-SP1 antibody was added to the substrate and incubated for 1 hour to attach the monoclonal MT-SP1 antibody with the protein A/G. ELISA was performed to confirm the attachment of the monoclonal MT-SP1 antibody. In detail, 50 μl of 100 ng/ml of MT-SP1 antigen was incubated on the substrate for 1 hr, followed by additional hour of incubation after adding poly MT-SP1 antibody conjugated with gold nanoparticles. Absorbance was measured by using ELISA reader after performing a silver enhancement method, and measured results are shown in FIG. 6. Referring to FIG. 6, there was an increase in absorbance depending on the concentration of protein A/G. This result suggests that there was a selective fixation of protein A/G by UV irradiation. As shown in the image in the bottom right corner of FIG. 6, the left substrates are shown as black color due to silver enhancement after UV irradiation. However, the right substrates are shown as transparent color since no selective antibody fixation occurred due to no UV irradiation which led to no silver enhancement.

### Example 4

### Analysis of the Fixation Level of Biomaterials Depending on the Intensity of UV Irradiation

Five experimental samples were prepared to analyze the antibody fixation. In detail, the BSA-diazirine was immobilized on five identical 96-well plate substrates (first to fifth substrates) by incubating the BSA-diazirine prepared in Example 1 on the substrates for 30 minutes. Unbound BSA-diazirine was removed by washing with 0.01% Tween-20, and the substrates were dried by blowing N₂ gas across the substrates. 50 μl of protein A/G at the concentration of 1 mg/ml was added to the surface of each substrate. Each substrate was irradiated with UV light (wave length of 365 nm) for 15 min at an UV intensity ranging from about 5 to 18 W/m². The absorbance was measured by following the experimental steps as describe in Example 3. The results are shown in FIG. 7. As shown in FIG. 7, there was a rapid increase in absorbance at 9 W/m² and then the slope became gentle.

### Example 5

### Analysis of the Fixation Level of Biomaterials Depending on the Duration of UV Irradiation

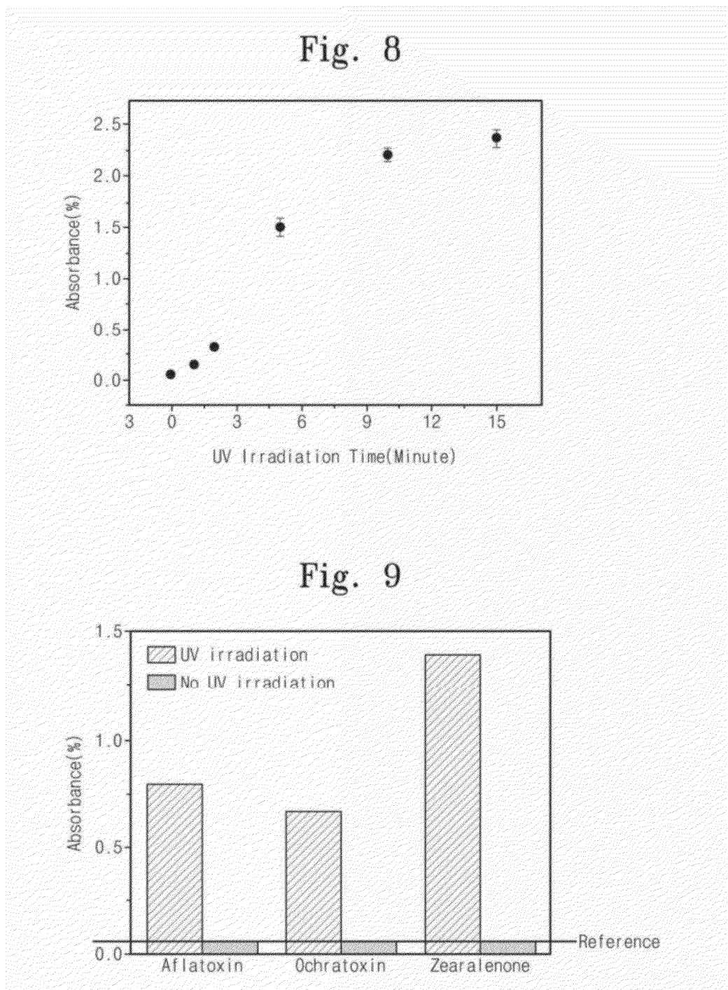

Six experimental samples were prepared to analyze the antibody fixation. In detail, the BSA-diazirine was immobilized on six identical 96-well plate substrates (first to sixth substrates) by incubating the BSA-diazirine prepared in Example 1 on the substrates for 30 minutes. Unbound BSA-diazirine was removed by washing with 0.01% Tween-20, and the substrates were dried by blowing N₂ gas across the substrates. 50 μl of protein A/G at the concentration of 1 mg/ml was added to the surface of each substrate. No UV light was irradiated on the first substrate. UV light (wavelength of 365 nm) was irradiated on the second to sixth substrates at the intensity of 8 W/m². The duration of UV irradiation varied from 1 to 15 minutes. Absorbance was measured by following the experimental steps as described in Example 3. The results are shown as a graph in FIG. 8. As shown in FIG. 8, there was an increase in absorbance at 6 minutes of UV irradiation, and then the slope became gentle. The results suggest that 10 minutes are the optimal length of time for UV irradiation, and there is a possibility of decrease in absorption when the irradiation time is longer than 15 minutes.

### Example 6

### Fixing Three Types of Food Toxins

There are three types of food toxins: aflatoxin, ochratoxin, and zearalenone. Unlike membrane type serine protease 1 (MT-SP1), the molecular weight of these food toxins are small and no polyclonal antibodies are available. Therefore, it is difficult to detect these toxins using a sandwich ELISA method. Experiments were performed to detect these toxins.

First, an experiment was carried out on aflatoxin. The BSA-diazirine prepared from Example 1 was added to a 96-well plate substrate and incubated for 30 minutes to immobilize the BSA-diazirine on the substrate. Unbound BSA-diazirine was removed by washing with 0.01% Tween-20, and the substrate was dried by blowing N₂ gas across the substrate. 50 μl of Aflatoxin at the concentration of 500 μg/ml was added to the substrate and irradiated with UV light for 15 minutes at the intensity of 18 W/m². Absorbance was measured using a sliver enhancement method and gold nano particle conjugates. The measured absorbance was 0.8% under UV irradiation. The absorbance of aflatoxin was 0.06% under same experimental condition without UV irradiation.

Secondly, an experiment was carried out on ochratoxin. The BSA-diazirine prepared in Example 1 was added to a 96-well substrate and incubated for 30 minutes to immobilize the BSA-diazirine on the substrate. Unbound BSA-diazirine was removed by washing with 0.01% Tween-20, and the substrate was dried by blowing N₂ gas across the substrate. 50 μl of Ochratoxin at the concentration of 1 mg/ml was added to the substrate and irradiated with UV light for 15 minutes at the intensity of 18 W/m². Absorbance was measured using a sliver enhancement method and gold nano particle conjugates. The absorbance was 0.67% under UV irradiation. The absorbance of Ochratoxin was 0.05%, under same experimental condition without UV irradiation.

Thirdly, an experiment was carried out on zearalenone. The BSA-diazirine prepared in Example 1 was added to a 96-well substrate and incubated for 30 minutes to immobilize the BSA-diazirine on the substrate. Unbound BSA-diazirine was removed by washing with 0.01% Tween-20, and the substrate was dried by blowing N₂ gas across the substrate. 50 μl of Zearalenone at the concentration of 500 μg/ml was added to the substrate and irradiated with UV light for 15 minutes at the intensity of 18 W/m². Absorbance was measured using a sliver enhancement method and gold nano particle conjugates. The absorbance was 1.4% under UV irradiation. The absorbance of Zearalenone was 0.06%, under same experimental condition without UV irradiation.

The results from three types of food toxins are shown in FIG. 9. As shown in FIG. 9, the food toxins can be selectively fixed to BSA-diazirine depending on UV irradiation.

The silver enhancement method used in the examples is as follows.

1) Lights are turned off after setting up experiment reagents.

2) An adequate amount of silver enhancer kit is dispensed into each test tube (150 μl of AgNO₃ solution and 150 μl of sodium citrate solution will be dispensed in each well. Contact of pipette tip with the bottle should be avoided to prevent contamination of the solution).

3) Add 150 μl of sodium citrate solution to each well (the order of adding AgNO₃ solution and sodium citrate solution is interchangeable. For example, sodium citrate solution is added first).

4) Add 150 μl of sodium citrate solution into each well and start the timer (12 minutes 30 seconds, time can vary depending on experimental conditions).

5) Carefully cover a 96-well substrate with an aluminum foil (to prevent contamination).

6) Prepare distilled water in a washing container and once the reaction is completed, rinse the 96-well substrate with distilled water before drying with N₂ gas (only the color of the well should change but not the solution).

### Example 7

### Forming a Micro Pattern by Selective Antibody Fixation

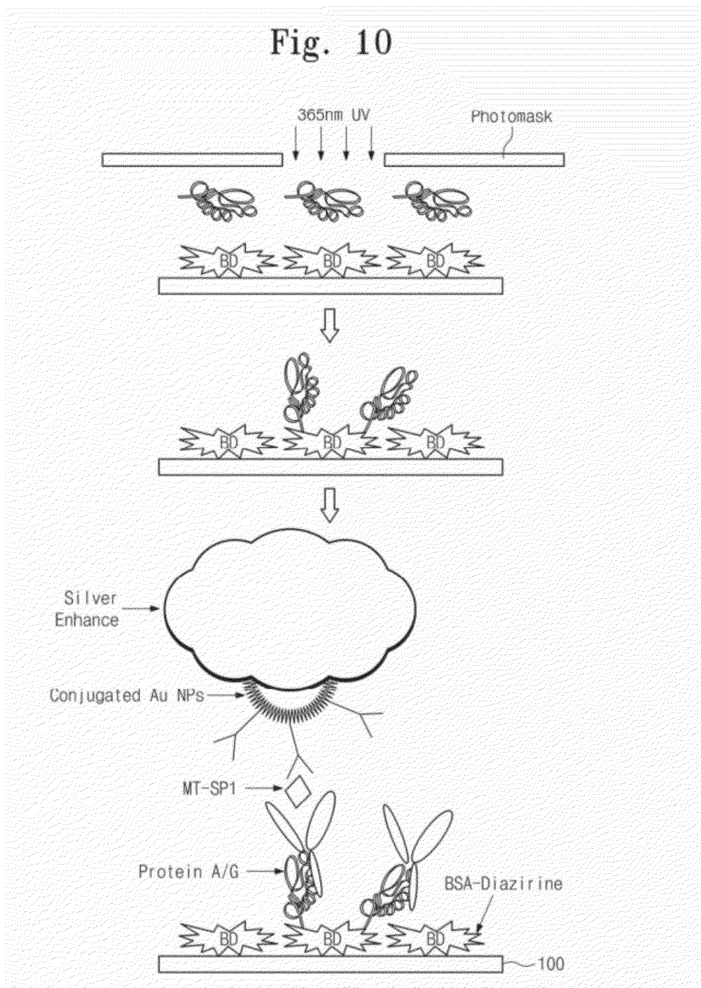

In this Example, antibody was fixed according to the method described in Example 2; however, a micro pattern was formed by selectively casting the UV light using a photomask. A process for selective fixation is illustrated in FIG. 10. Referring to FIG. 10, protein A/G is fixed to the region where UV was cast through a photomask. Antibody attached to the immobilized protein A/G was confirmed by a sandwich ELISA method and a silver enhancement method.

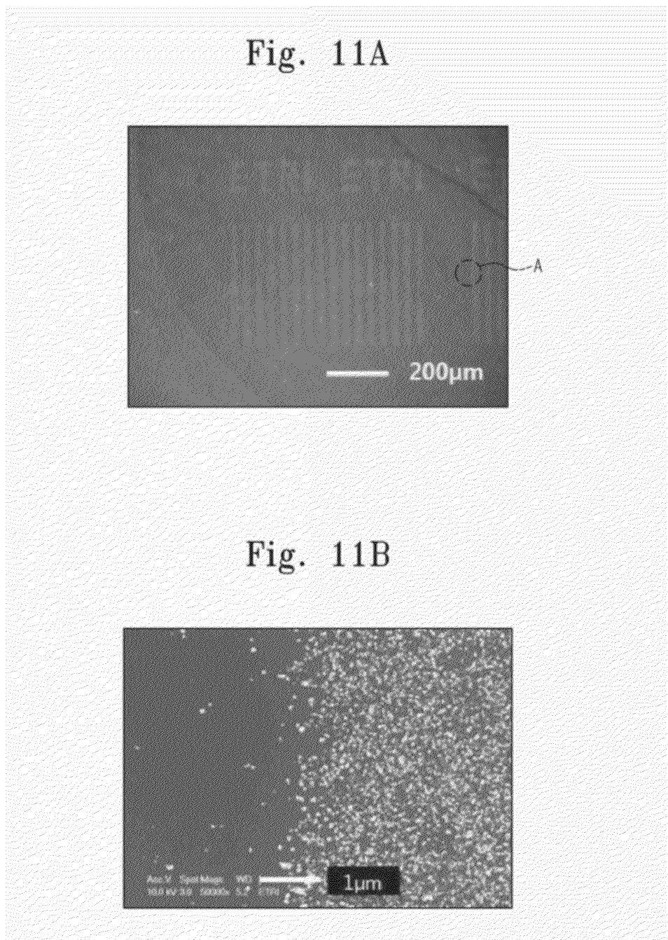

FIGS. 11A and 11B illustrate images of a micro pattern formed in Example 7. FIG. 11B is an enlarged image of A region (boarder region) of an FE-SEM image of FIG. 11A. The size of characters in FIG. 11A is about 20 μm. According to Example 7, the antibody was selectively fixed at desired locations within a distance of about 20 μm by selective UV irradiation.

In some embodiments of the present invention, the bovine serum albumin-diazirine may function as a blocker that prevents non-specific binding, as well as a linker which links a solid support and a biomaterial by photoreaction (by UV irradiation). That is, a biomaterial can be selectively fixed using the bovine serum albumin-diazirine.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A bovine serum albumin-diazirine, in which bovine serum albumin having an amine group is covalently bonded to at least one NHS-diazirine (N-Hydroxysuccinimide diazirine) having a structure of Formula 1.

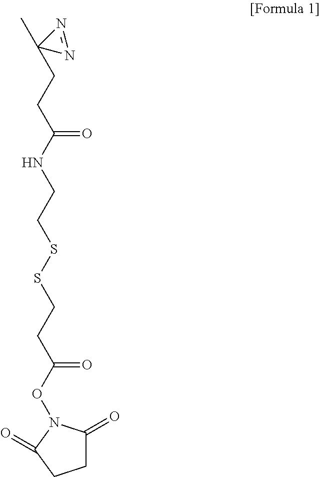

2. A method for preparing a bovine serum albumin-diazirine, the method comprising:
allowing bovine serum albumin containing an amine group to react with Sulfo-SDAD (sulfosuccinimidyl 2-([4,4′-azipentanamido]ethyl)-1,3′-dithioproprionate) having a structure of Formula 2 so as to obtain a bovine serum albumin-diazirine in which at least one NHS-diazirine having a structure of Formula 1 is covalently bonded to the amine group of the bovine serum albumin.

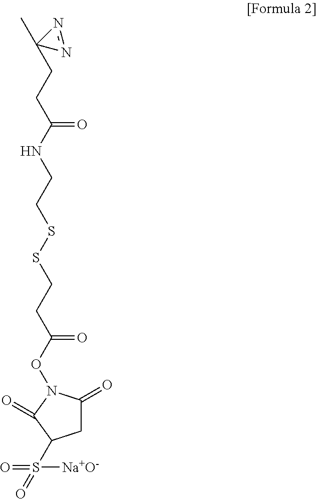

3. The method of claim 2, wherein the allowing of the bovine serum albumin to react with the Sulfo-SDAD comprises stirring the bovine serum albumin and the Sulfo-SDAD.

4. The method of claim 2, wherein after the allowing of the bovine serum albumin to react with the Sulfo-SDAD, the method further comprises removing remaining bovine serum albumin and Sulfo-SDAD.

5. A method for fixing a biomaterial, the method comprising:
immobilizing the bovine serum albumin-diazirine of claim 1 on a substrate; and
selectively fixing a biomaterial to the bovine serum albumin-diazirine by UV irradiation.

6. The method of claim 5, wherein the immobilizing of the bovine serum albumin-diazirine comprises incubating the bovine serum albumin-diazirine on the substrate.

7. The method of claim 5, wherein the selective fixing of the biomaterial comprises:
selectively fixing protein A/G by UV irradiation; and
fixing a biomaterial to the selectively fixed protein A/G.

8. The method of claim 7, wherein in the selective fixing of the biomaterial, the UV irradiation is performed for about 6 minutes to about 15 minutes.

9. The method of claim 7, wherein in the selective fixing of the biomaterial, the UV irradiation is performed with UV intensity of about 5 W/m² to about 18 W/m².

10. The method of claim 5, wherein the selective fixing of the biomaterial comprises selectively casting UV light using a photomask.

11. The method of claim 10, further comprising selectively casting UV light to form a micro pattern.

12. The method of claim 5, wherein the biomaterial comprises at least one selected from the group consisting of antibodies, membrane type serine proteases, and food toxins.
